(19) United States
(12) Patent Application Publication
(10) Pub. No.: US 2013/0309172 A1
(43) Pub. Date: Nov. 21, 2013

(54) CLEAVABLE FUNCTIONALIZED NANOPARTICLES

(71) Applicant: City Of Hope, (US)

(72) Inventors: Jacob M. Berlin, Monrovia, CA (US); Anil K. Suresh, Monrovia, CA (US); John Williams, Monrovia, CA (US)

(73) Assignee: City of Hope, Duarte, CA (US)

(21) Appl. No.: 13/888,306

(22) Filed: May 6, 2013

Related U.S. Application Data
(60) Provisional application No. 61/642,973, filed on May 4, 2012.

(51) Int. Cl. A61K 9/50; A61K 49/00

(52) U.S. Cl. CPC A61K 9/50; A61K 49/0004
USPC 424/9.1; 424/490

(57) ABSTRACT

Provided herein, inter alia, are compositions of functionalized nanoparticles and methods of using functionalized nanoparticles in treating, imaging, and/or detecting cancers.

### CROSS-REFERENCES TO RELATED APPLICATIONS

This application claims priority to Provisional Application No. 61/642,973 filed May 4, 2012.

### BACKGROUND OF THE INVENTION

Targeted delivery of therapeutic agents to tumor sites increases efficacy and limits off-target toxicity (Arap 1998; Vasir 2005). Nanoparticles are an emerging class of targeted drug delivery systems (Ferrari 2005; Riehemann 2009; Berlin 2010). However, despite advances in using nanoparticles as targeted drug delivery systems, most constructs still predominantly accumulate in the liver (Bhirde 2009; Harrington 2000) therefore causing undesired side-effects. Accordingly, there is a need in the art for more effective methods and compositions for delivering nanoparticles to treatment sites. Provided herein are solutions to these and other problems in the art.

### BRIEF SUMMARY OF THE INVENTION

Provided herein, inter alia, are functionalized nanoparticles. In one aspect, a functionalized nanoparticle including a nanoparticle core and a nanoparticle coating is provided. The nanoparticle core is about 2 to about 35 nm in length. The nanoparticle coating includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

In another aspect, a plurality of functionalized nanoparticles within a vessel is provided. Each functionalized nanoparticle includes a nanoparticle core and a nanoparticle coating. The plurality of nanoparticle cores has an average particle of size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

In another aspect, a pharmaceutical composition including a plurality of functionalized nanoparticles is provided. Each functionalized nanoparticle includes a nanoparticle core, a therapeutic agent bonded to the nanoparticle core and a nanoparticle coating. The plurality of nanoparticle cores has an average particle of size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

In another aspect, a method of treating cancer is provided. The method includes administering a plurality of functionalized nanoparticles within a vessel to a subject in need thereof. Each functionalized nanoparticle includes a nanoparticle core, an anti-cancer agent bonded to the nanoparticle core and a nanoparticle coating. The plurality of nanoparticle cores has an average particle size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

In another aspect, a method of detecting a cancer cell within a subject is provided. The method includes administering a plurality of functionalized nanoparticles within a vessel to a subject in need thereof. Each functionalized nanoparticle includes a nanoparticle core and a nanoparticle coating. The plurality of nanoparticle cores has an average particle size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site. The method includes allowing a portion of the functionalized nanoparticles to localize to the cancer cell and allowing a cleaving agent to cleave the cleavage site of at least one of the functionalized nanoparticles of the portion thereby removing the nanoparticle coating and forming a denuded detectable nanoparticle. The method further includes detecting the denuded detectable nanoparticle thereby detecting the cancer cell.

### BRIEF DESCRIPTION OF THE DRAWINGS

FIG. 1: Characterization of AuNPs wherein (A) shows UV-vis spectroscopy measurement of AuNPs, (B) shows Dark-field and (C) shows transmission electron microscopy images of the AuNPs, and (D) shows Histogram plot of AuNP size distribution analyzed from TEM images.

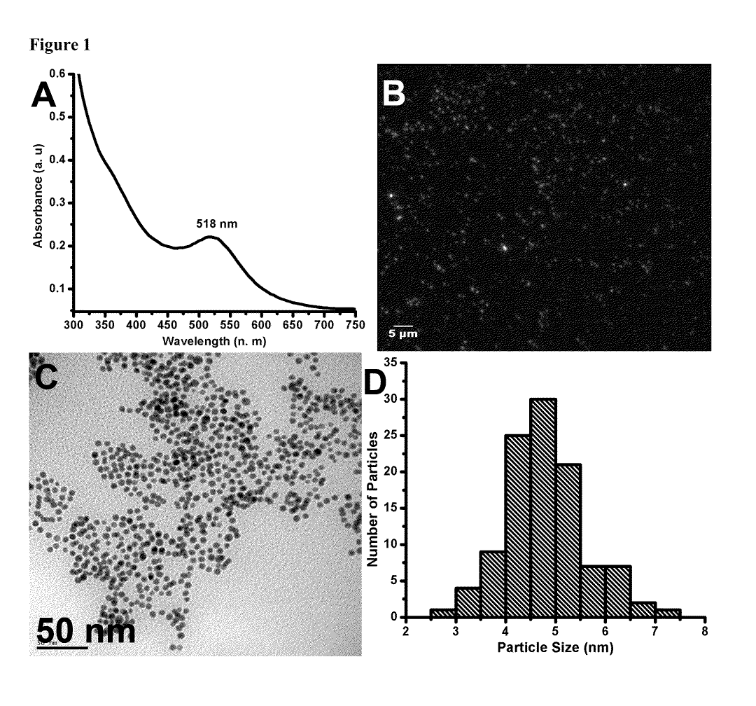

FIG. 2: Selective denuding of engineered AuNPs by MMP wherein (A) shows functionalization of AuNPs and removal of PEG blocking element by MMP, (B) shows Illustration of long-term goal of tumor targeting by AuNPs (MMPs in the tumor environment may cleave the responsive PEG coating, denuding the AuNPs and leading to their uptake by cells; In normal tissue, no such cleavage occurred and the PEGylated AuNPs may continue to circulate).

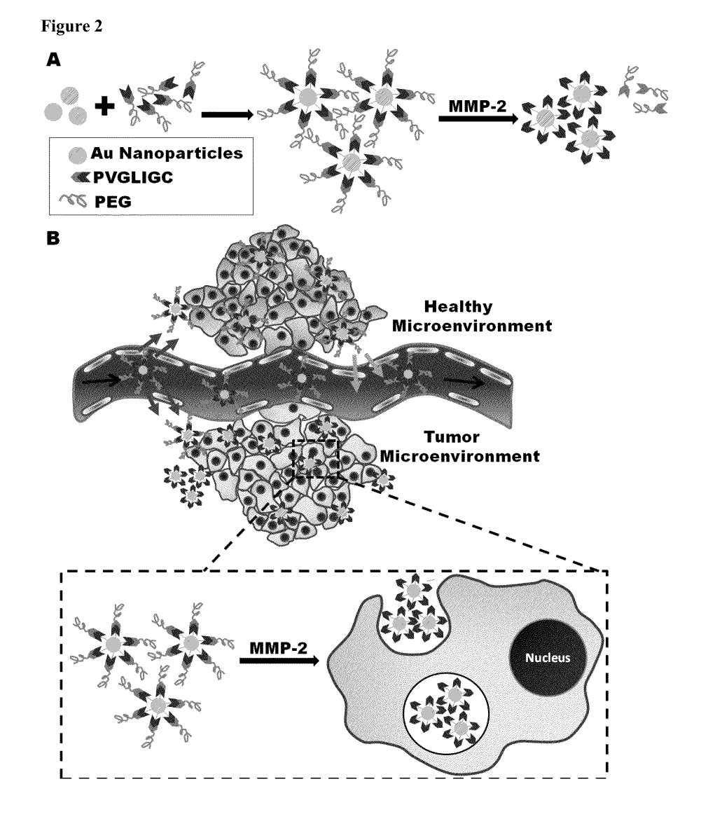

FIG. 3: Characterization of AuNP conjugates before and after cleavage wherein (A) shows UV-vis spectroscopy measurements of cleavable AuNP before and after cleavage by MMP, AuNP alone (positive control), and denuded control AuNP (negative control), Representative TEM images of cleavable AuNPs before (B) and after (C) cleavage by MMP and denuded control AuNPs (D), FFF analysis of cleavable AuNPs before (E) and after (F) cleavage by MMP and denuded control AuNPs(G).

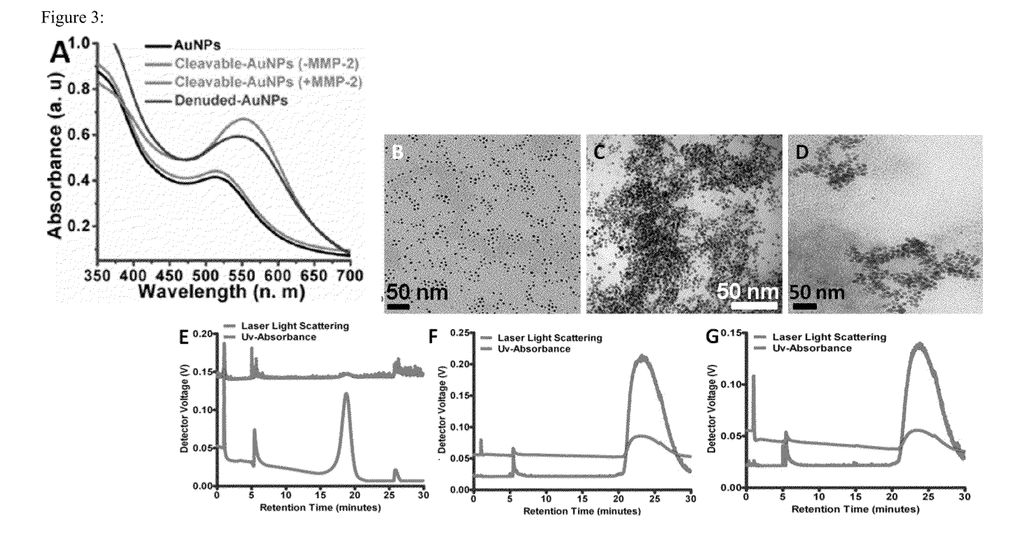

FIG. 4: Characterization of AuNP conjugate controls before and after cleavage wherein (A) shows UV-vis spectroscopy measurements of non-cleavable AuNP before and after cleavage by MMP, cleavableAuNP after exposure to trypsin, AuNP alone (positive control), and blocker control before and after cleavage, Representative TEM images of (B) non-cleavable AuNPs and (C) blocker control AuNPs after exposure to MMP. (D) Dark-field images of cleavable AuNP before (D) and after (E) cleavage and denuded AuNP control (F) (Aggregates can be clearly seen in (E) and (F)).

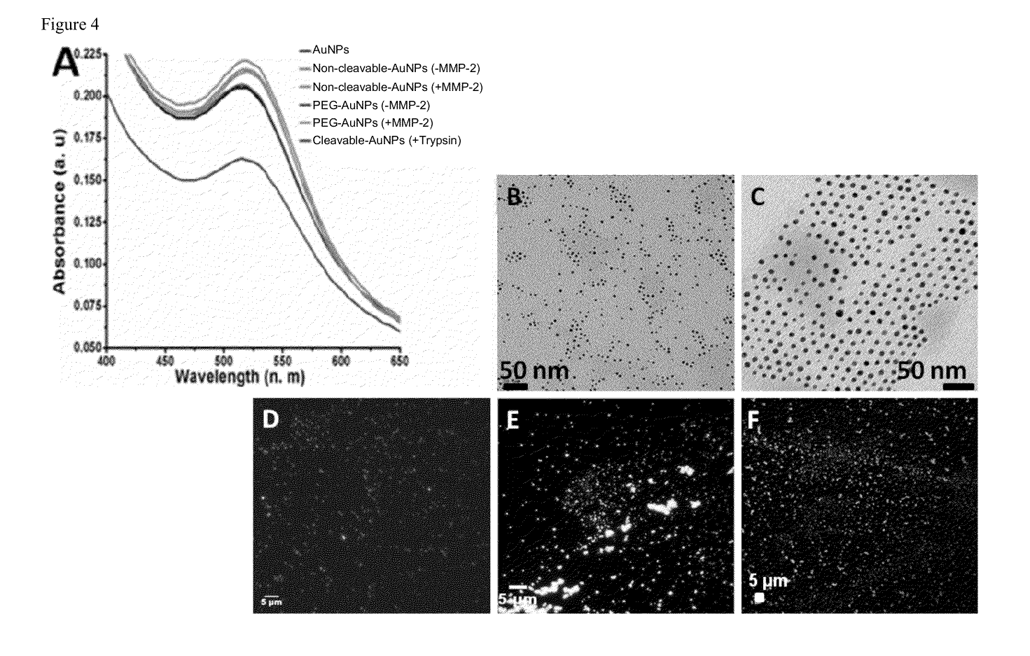

FIG. 5: Field flow fractionation analysis of various AuNP conjugates: blocker control (A) and non-cleavable AuNP before (B) and after (C) exposure to MMP-2.

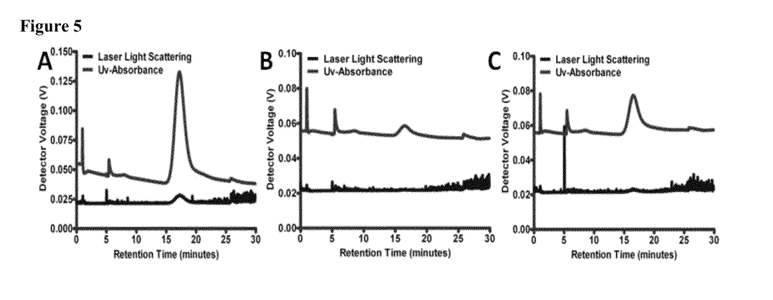

FIG. 6: In vitro cytotoxicity evaluation for cleavable AuNPs and denuded control AuNPs wherein (A) shows Cell viability as measured by MTS assay, LIVE/DEAD staining and imaging for cells treated with (B) nothing, (C) cleavable AuNPs, and (D) denuded control AuNPs (Green fluorescence indicates live cells, and dead or compromised cells are stained red: Error bars indicate standard deviation. * p<0.05 vs. control).

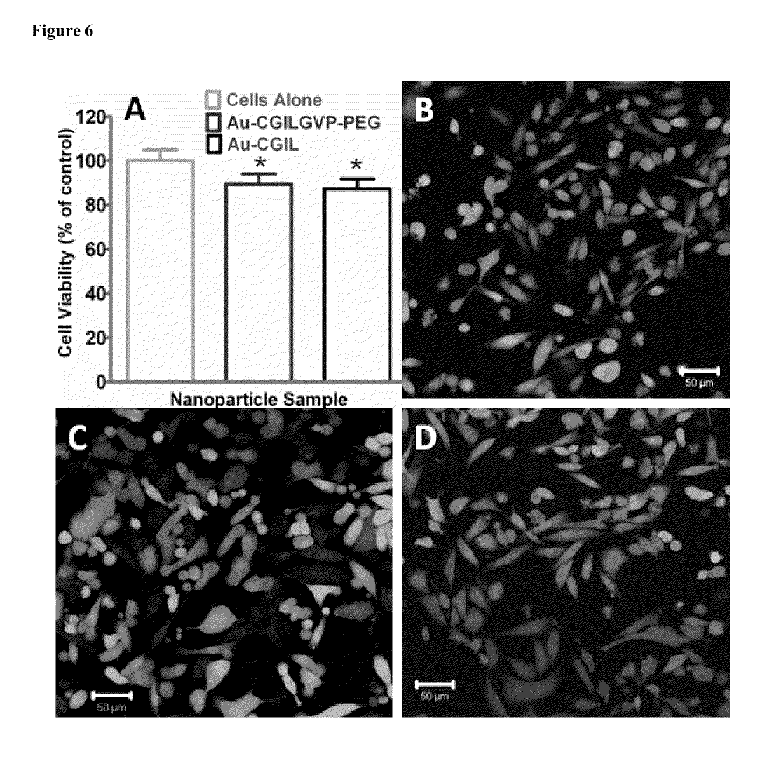

FIG. 7: In vitro cytotoxicity evaluation for various AuNP conjugate control samples wherein (A) shows Cell viability as measured by MTS assay, LIVE/DEAD staining and imaging for cells treated with (B) AuNPs, (C) blocker control AuNPs, and (D) non-cleavable AuNPs (Green fluorescence indicates live cells, and compromised cells are stained red: Error bars indicate standard deviation. * p<0.05 vs. control).

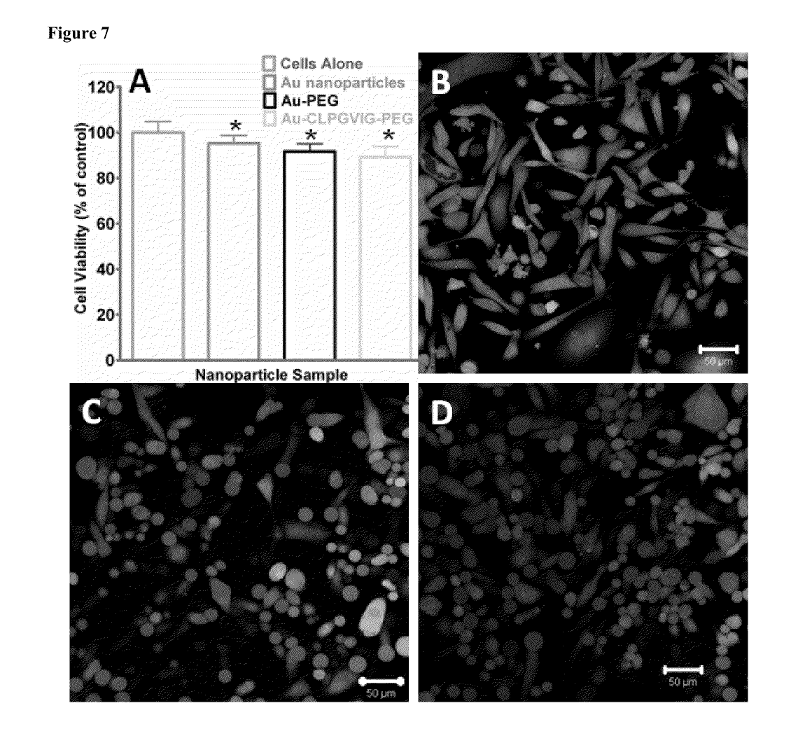

FIG. 8: Hyperspectral mapping of AuNP cell uptake on dark-field microscopy images wherein (A) shows Dark-field image of AuNPs alone (Inset shows the hyperspectrallibrary derived from this image that was used to map the subsequent images), Hyperspectral mapping of AuNPs (false-colored) on dark-field microscopy images of MDA-MB-231 cells either treated with cleavable AuNPs (B) before and (C) after exposure to MMP-2 or (D) denuded control AuNPs.

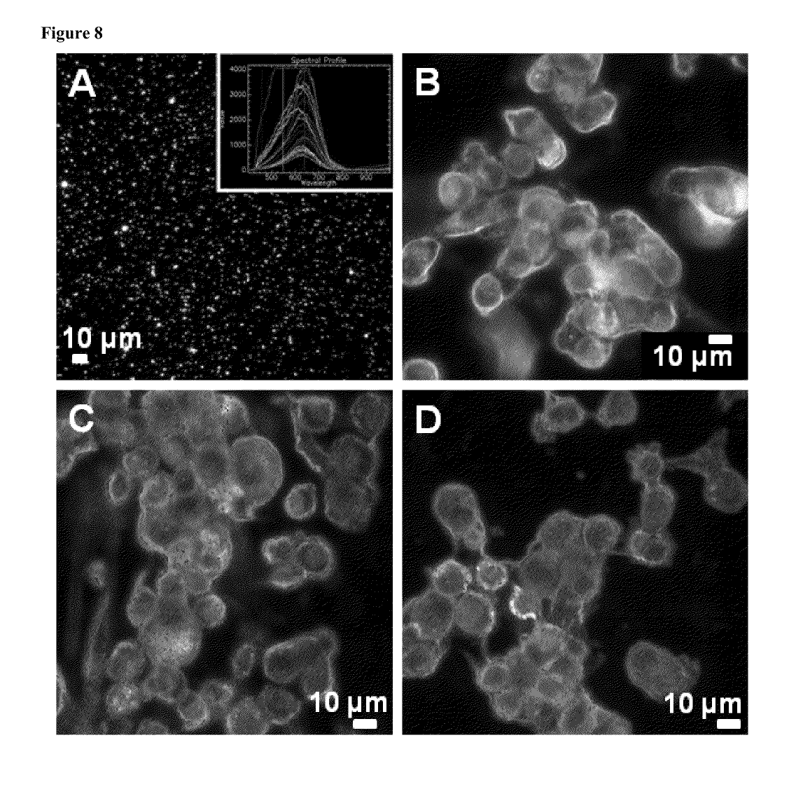

FIG. 9: Hyperspectral mapping of dark-field microscopy images of MDA-MB-231 untreated control cells (A) and cells treated with blocker control (B), non-cleavable AuNPs before (C) and after (D) exposure to MMP-2, and AuNPs alone (E).

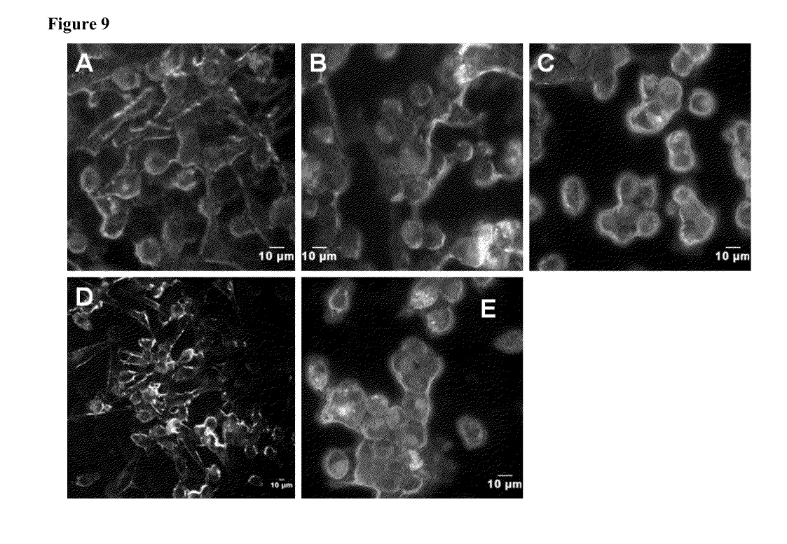

FIG. 10: TEM imaging of uptake and intracellular localization of AuNPs showing TEM images of MDA-MB-231 cells treated with cleavable AuNPs before (A, B, and C) and after (D, E, and F) exposure to MMP-2 or denuded control AuNPs (G, H and I) shown at increasing magnifications (Arrows indicate aggregates of gold nanoparticles).

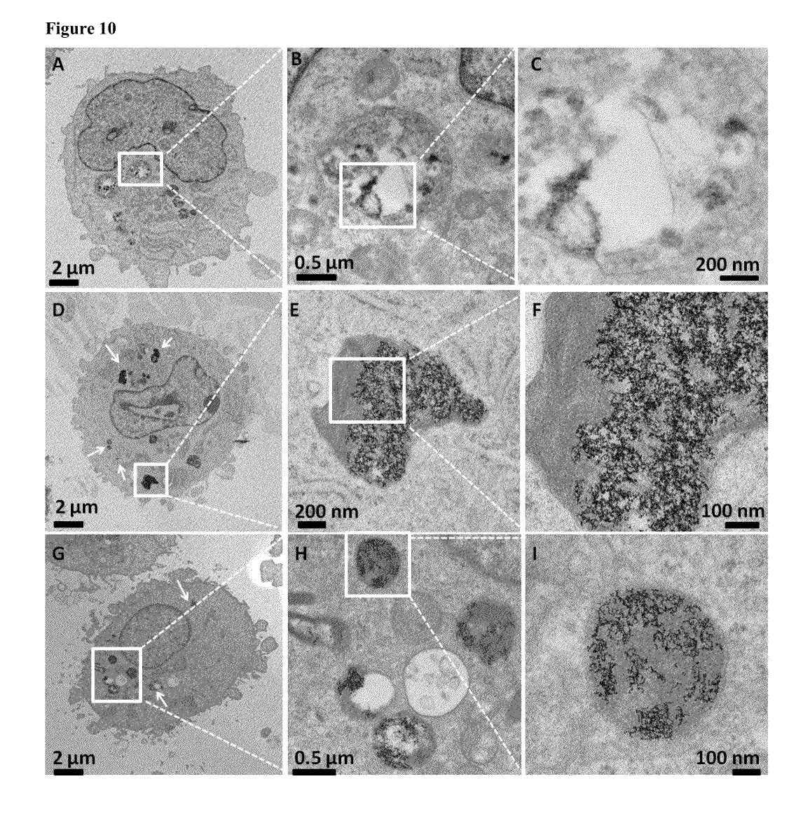

FIG. 11: Uptake and intracellular localization based on ultra-microtome followed by TEM imaging showing TEM images of MDA-MB-231 untreated control cells (A and B) and cells treated with blocker control AuNPs (C and D), non-cleavable AuNPs (E and F), and AuNP alone (G and H) at increasing magnifications (Arrows indicate aggregates of gold nanoparticles).

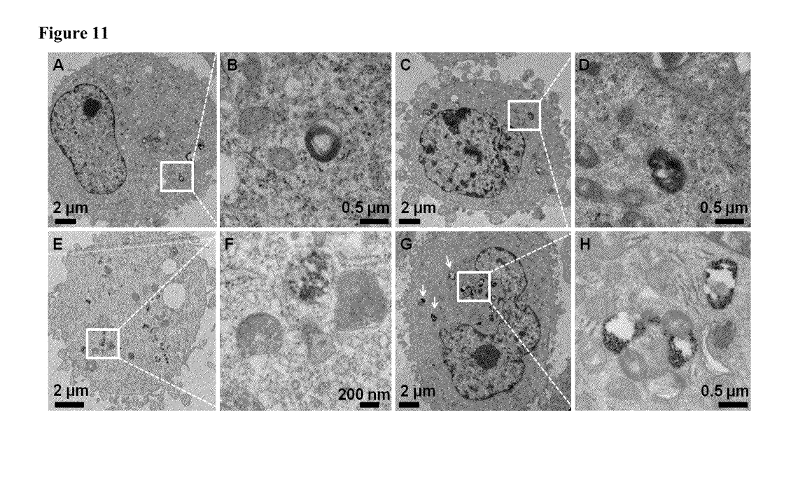

FIG. 12: Higher magnification TEM images of MDA-MB-231 cells treated with cleavable AuNPs exposed to MMP-2 (Images were taken from different regions of the grid and from different cells to show the widespread uptake of the AuNPs).

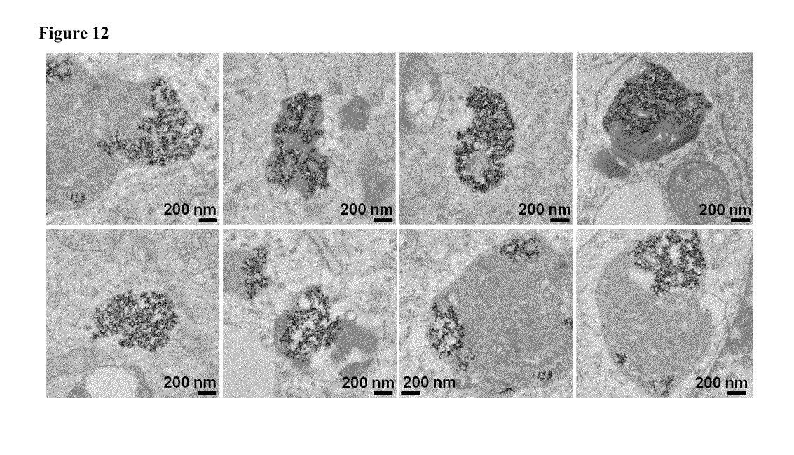

FIG. 13: Uptake and intracellular localization based on ultra-microtome followed by TEM imaging showing TEM images of fibroblast untreated control cells (A and B) and cells treated with blocker control (C and D), cleavable AuNPs before (E and F) and after (G and H) exposure to MMP-2, non-cleavable AuNPs (I and J), AuNPs alone (K and L), and denuded control AuNPs (M and N) at increasing magnifications (Arrows indicate aggregates of gold nanoparticles).

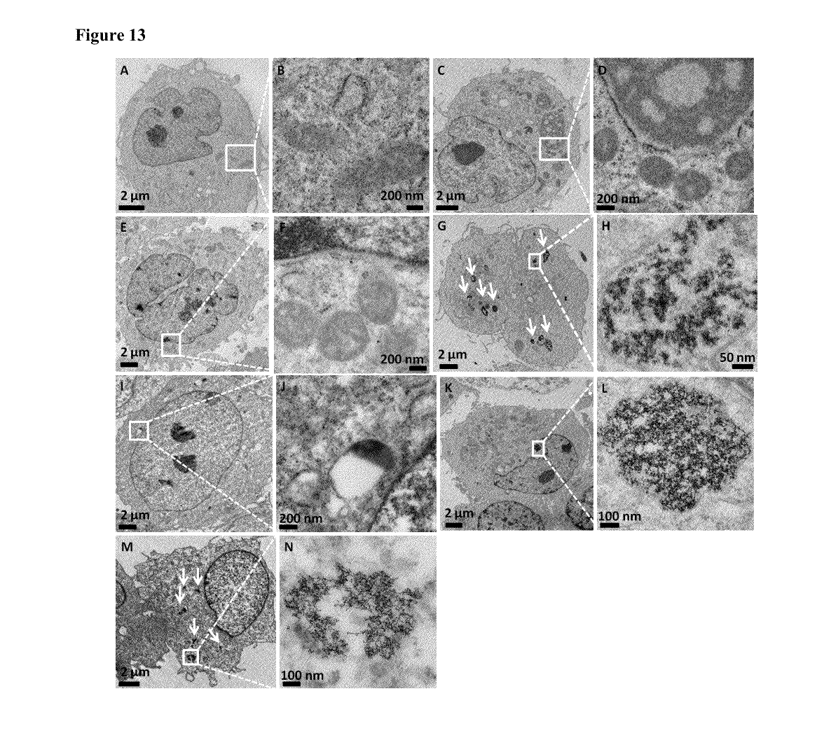

FIG. 14: Characterization of various Au-conjugate controls wherein (A) shows UV-vis spectra of the various positive and negative control samples used, field flow fractionation analysis of the various Au-conjugates of PEG-AuNPs (B), non-cleavable AuNPs before (C) and after (D) exposure to MMP-2, (E) shows box plot analysis of the hydrodynamic size distributions (nm) of the different AuNP conjugates obtained from dynamic light scattering measurements. (S2)

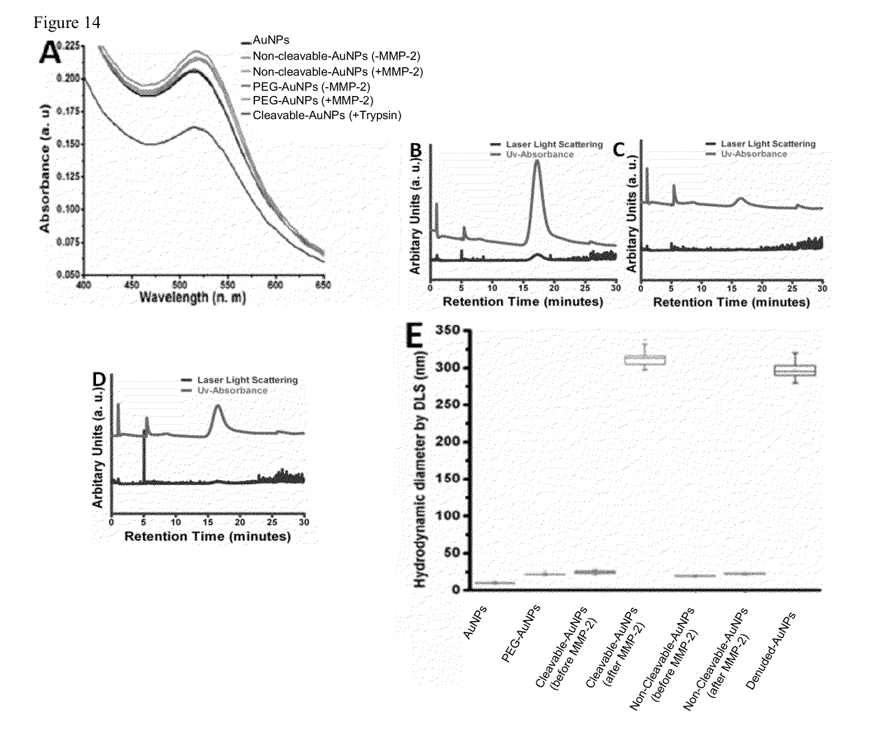

FIG. 15: Thermgravimetric analysis (TGA) of the cleavable-AuNP conjugates shown in the form of percent (%) weight loss against a range of temperatures. (S3)

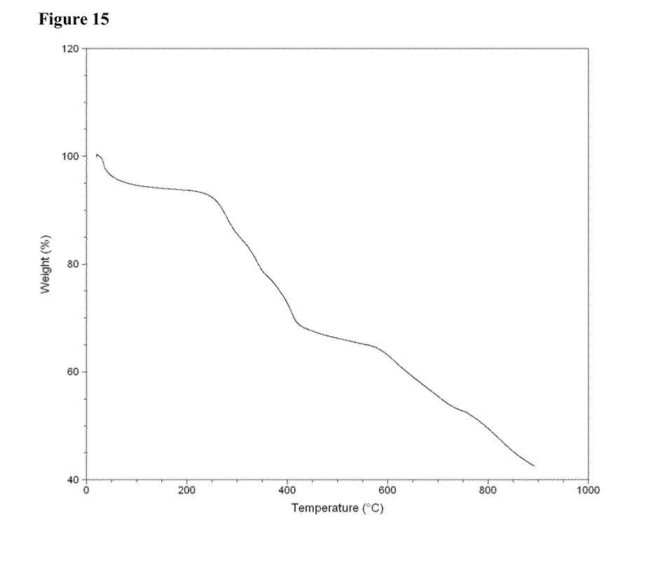

FIG. 16: In Vitro cytoxicity evaluation for various AuNPs control samples wherein (A) shows Cell viability as measured by MTS assay, LIVE/DEAD staining and imaging for cells treated with (B) AuNPs, (C) PEG-AuNPs and, (D) non-cleavable AuNPs in the absence and (E) presence of MMP-2 (Green fluorescence indicates live cells, and compromised cells are stained red: The error bars indicate the standard deviation: * p<0.05 relative to control). (S4)

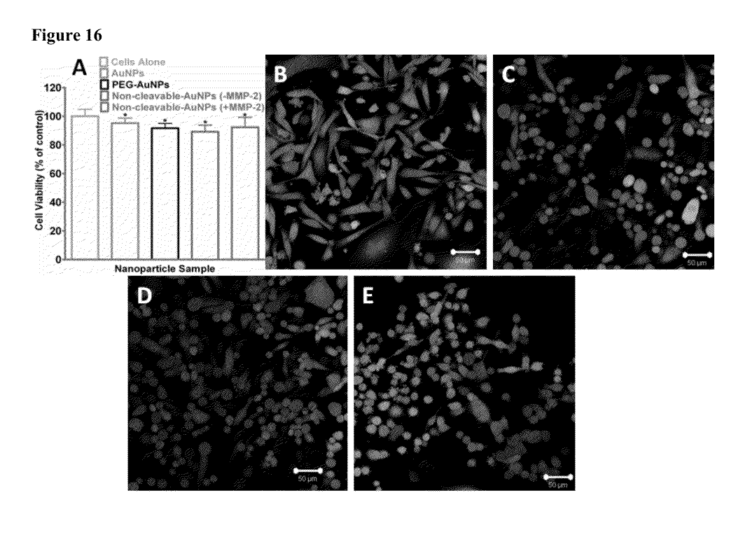

FIG. 17: Hyperspectral mapping of dark-field microscopy images of the MDA-MB-231 untreated control cells (A) and the cells treated with the various conjugates; PEG-AuNPs (B), non-cleavable AuNPs before (C) and after (D) treatment with MMP-2 [15 μg/mL]. (S5)

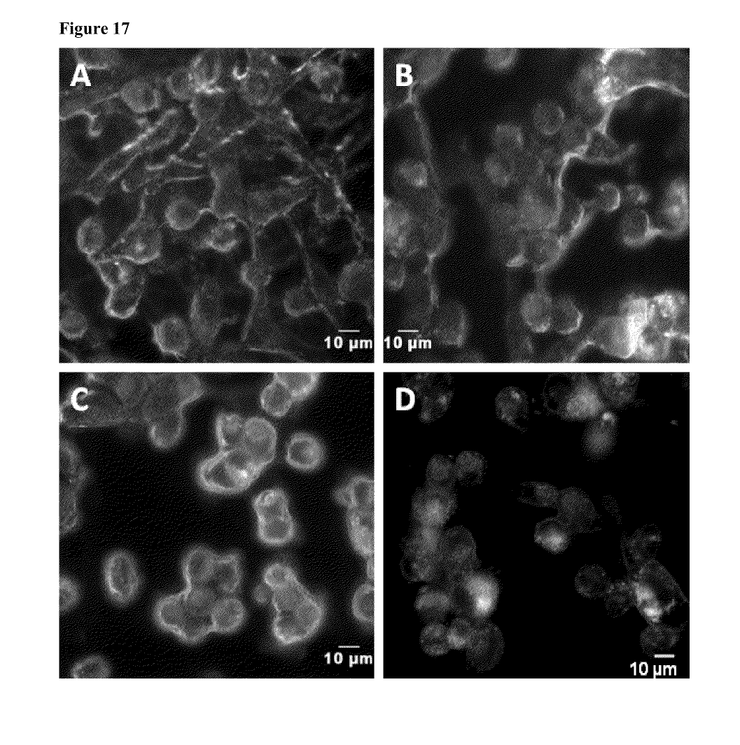

FIG. 18: TEM images of the MDA-MB-231 control cells (A and B) and the cells treated with the PEG-AuNPs (C and D), non-cleavable AuNPsalone (E and F) and in the presence of MMP-2 (G and H) at increasing magnifications. (S6)

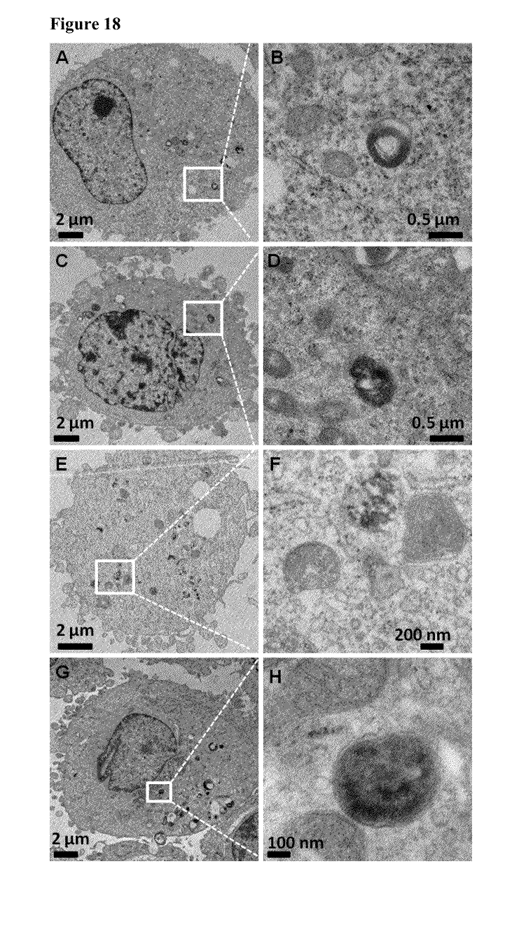

FIG. 19: Viability analysis of MDA-MB-231 cells treated with various AuNP conjugates obtained from confocal microscopy images using Image J (Version 1.4.3) software. (table s1)

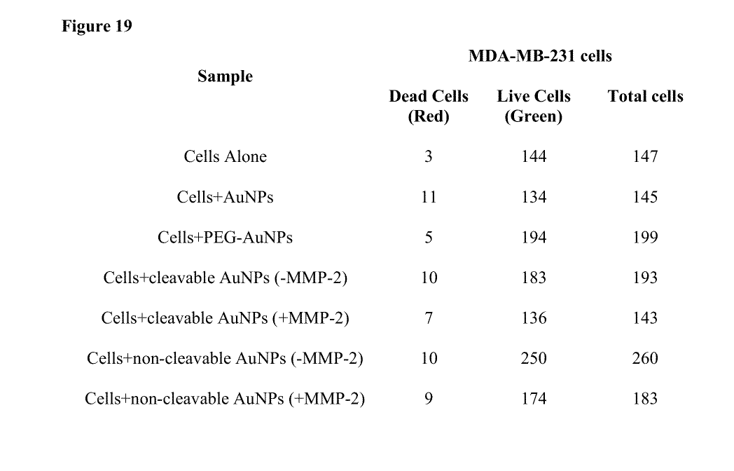

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, S, Se and Si, and wherein the nitrogen, selenium, and sulfur atoms may optionally be oxidized, and the nitrogen heteroatom may optionally be quaternized. The heteroatom(s) O, N, P, S, Se, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SeR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g. 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one (e.g. from one to four) heteroatoms (e.g. N, O, or S), wherein sulfur heteroatoms are optionally oxidized, and the nitrogen heteroatoms are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substitutents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)N R′R″, —NR″C(O)R′, —NR′—C(O)NR″R′″, —NR″C(O)₂R′, —NR—C(NR′R″R′″═NR″″, —NR—C(NR′R″)═NR′″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC (O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

The nanoparticle core may be attached to the cleavage site through a nanoparticle binding moiety. The nanoparticle binding moiety may be a divalent linker formed by reacting a functional (reactive) group attached to the cleavage site with the nanoparticle core (e.g. a functional (reactive) group on the nanoparticle core). Currently favored classes of reactions available with reactive known reactive groups are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(i) metal silicon oxide bonding; and

(l) metal bonding to reactive phosphorus groups (e.g. phosphines) to form, for example, phopshate diester bonds.

The reactive functional groups can be chosen such that they do not participate in, or interfere with, the chemical stability of the functionalized nanoparticles described herein. Alternatively, a reactive functional group can be protected from participating in the crosslinking reaction by the presence of a protecting group.

Linkers may also be employed between the nanoparticle binding moiety and the cleavage site and/or between the cleavage site and the water soluble moiety. Linkers may include reactive groups at the point of attachment to of the nanoparticle binding moiety, cleavage site and/or the mobile detectable analyte binding reagents. Any appropriate linker may be used in the present invention, including substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers nucleic acids that have the same basic chemical structure as a naturally occurring nucleic acids. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

“Synthetic mRNA” as used herein refers to any mRNA derived through non-natural means such as standard oligonucleotide synthesis techniques or cloning techniques. Such mRNA may also include non-proteinogenic derivatives of naturally occurring nucleotides. Additionally, “synthetic mRNA” herein also includes mRNA that has been expressed through recombinant techniques or exogenously, using any expression vehicle, including but not limited to prokaryotic cells, eukaryotic cell lines, and viral methods. “Synthetic mRNA” includes such mRNA that has been purified or otherwise obtained from an expression vehicle or system.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The word “protein” denotes an amino acid polymer or a set of two or more interacting or bound amino acid polymers.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

Lengths and sizes of nanoparticles and functionalized nanoparticles as described herein are measured using Transmission Electron Microscopy. All exemplified nanoparticle core length values reported herein are measured using the following protocol. For the transmission electron microscopy measurements the various gold nanoparticle samples were drop coated (5 μL) onto 200 mesh copper EM grids, air-dried and imaged using a FEI Tecnai 12 TEM equipped with a Gatan Ultrascan 2K CCD camera at an accelerating voltage of 120 kV. The average size distributions of the particles were obtained from the TEM images using Image J (version 1.4.3) software that were plotted using Origin Pro 8 software to obtain the histogram size distributions of the particles.

A “labeling agent” as used herein refers to a moiety that can be covalently or noncovalently attached to a compound or biomolecule that can be detected (e.g. quantitated) using techniques known in the art. In embodiments, the labeling agent is covalently attached. The labeling agent may provide for imaging of the attached nanoparticle. Exemplary detectable moieties are fluorophores, antibodies, reactive dies, radio-labeled moieties, magnetic contrast agents, and quantum dots. Exemplary fluorophores include fluorescein, BODIPY, and cyanine dyes. Exemplary radionuclides include Fluorine-18, Gallium-68, and Copper-64. Exemplary magnetic contrast agents include gadolinium, iron oxide and iron platinum, and manganese.

A “hydrophilic moiety” as used herein, refers to a nanoparticle binding moiety bonded to a nanoparticle core, a cleavage site covalently linked to the nanoparticle binding moiety, and a water soluble moiety covalently linked to the cleavage site. Exemplary hydrophilic moieties may be:

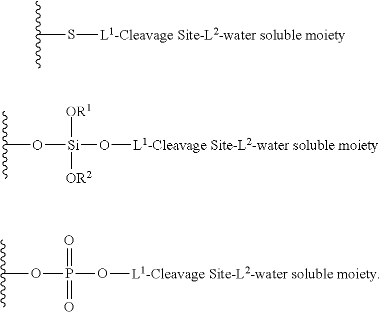

L¹, L², R¹, R², and R³ are as defined herein.

A “nanoparticle binding moiety” as used herein refers to chemical moiety attached at one point to the nanoparticle core and covalently attached at a second point to a linker.

A “cleavage site” as used herein, refers to a recognizable site for cleavage of the water soluble moiety from the nanoparticle core. In embodiments, the cleavage site is an amino acid sequence that is recognized and cleaved by a peptidase. Exemplary cleavage sites are defined herein.

A “water soluble moiety” as used herein refers to any moiety that enhances the water solubility of a nanoparticle to which it is bound or otherwise serves to prevent the functionalized nanoparticles from accumulating or aggregating. A water soluble moiety may alter the partitioning coefficient of a nanoparticle thereby making the nanoparticle more or less hydrophilic. Generally, greater hydrophobicity correlates to a higher partition constant. Likewise, generally, greater hydrophilicity correlates to a lower partition constant. In some embodiments, the water soluble groups can decrease the partition constant of precursor molecules (which have a higher partition constant before attachment of the water soluble group) at least by about 10%, 20%, 30%, 40%, 50%, 60%, 70%, 80%, or 90%. In some embodiments, the water soluble groups described herein can decrease the partition constant of the plurality of hydrophobicity moieties of the nanoparticle coating by 1-fold, 2-fold, 3-fold, 4-fold, or greater. Exemplary water soluble moieties include moieties (e.g. monovalent forms) such as poly(oxyethylated polyols) such as poly(oxyethylated glycerol), poly(oxyethylated sorbitol), and poly(oxyethylated glucose); poly(vinyl alcohol) (“PVA”); dextran; carbohydrate-based polymers and the like (including linear chains or branched chains); polyethylene glycol moieties of formula

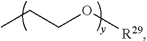

wherein y is an integer from 1 to 50 and R²⁹ is —OH or —OMe; polyvinylpyroolidone moieties; or poly 2-ethyl oxazoline moieties. Water soluble molecules may exist in polymeric form wherein at least two monomers are repeatedly covalently connected.

In some embodiments, the water soluble group can include a moiety containing a heteroatom (e.g., oxygen or nitrogen). In embodiments, the moiety contains an alcohol moiety (an organic moiety having an —OH bound to a carbon atom), ester linker moiety (the linker moiety —C(O)O— between two carbon atoms), ether linker moiety (the linker moiety —O— between two carbon atoms), amine (—NH₂) moiety, nitrile (—CN) moiety, ketone moiety (the linker moiety —C(O)— between two carbon atoms), or aldehyde (—C(O)H) moiety.

A “delivery agent” is used according to its ordinary meaning in the art and refers to chemical moiety bound to a nanoparticle that assists in delivering a functionalized nanoparticle, nanoparticle, denuded nanoparticle and the like to a particular site, such as a particular cell, organ or area within an organism. Thus delivery agents may assist in delivering therapeutic agents such as an anti-cancer agent and may additionally be detectable (e.g. a labeling agent).

The nanoparticles of the present invention may exist as salts, such as with pharmaceutically acceptable acids. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms are preferably regenerated by contacting the salt with a base or acid and isolating the parent in the conventional manner. The parent form differs from the various salt forms in certain physical properties, such as solubility in polar solvents.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease.

A “therapeutically effective amount” is an amount sufficient for an active to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulation”, “modulate”, or “modulator” are used in accordance with their plain ordinary meaning and refer to the act of changing or varying one or more properties. “Modulator” refers to a composition that increases or decreases the level of a target molecule or the function of a target molecule or the physical state of the target of the molecule. “Modulation” refers to the process of changing or varying one or more properties. For example, as applied to the effects of a modulator on a biological target, to modulate means to change by increasing or decreasing a property or function of the biological target or the amount of the biological target.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active or prodrug form of a nanoparticle as provided herein with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies, for example epigenetic inhibitors or multi-kinase inhibitors. The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g. to reduce metabolic degradation).

As used herein, the term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, metastatic bone cancer, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans).

### II. Compositions

In one aspect, a functionalized nanoparticle including a nanoparticle core and a nanoparticle coating is provided. The nanoparticle core may be about 2 to about 35 nm in length. The nanoparticle coating includes a plurality of hydrophilic moieties bonded to the nanoparticle core. Each of the hydrophilic moieties may include a nanoparticle binding moiety bonded to the nanoparticle core, a cleavage site covalently linked to the nanoparticle binding moiety, and a water soluble moiety covalently linked to the cleavage site.

Nanoparticles sizes and lengths as provided herein were measured using transmission electron microscopy (TEM) as follows. For the transmission electron microscopy measurements the various gold nanoparticle samples were drop coated (5 μL) onto 200 mesh copper EM grids, air-dried and imaged using a FEI Tecnai 12 TEM equipped with a Gatan Ultrascan 2K CCD camera at an accelerating voltage of 120 kV. The average size distributions of the particles were obtained from the TEM images using Image J (version 1.4.3) software that were plotted using Origin Pro 8 software to obtain the histogram size distributions of the particles.

The nanoparticle core may be about 2 nm to about 30 nm in length. The nanoparticle core may be about 2 nm to about 25 nm in length. The nanoparticle core may be about 2 nm to about 20 nm in length. The nanoparticle core may be about 2 nm to about 15 nm in length. The nanoparticle core may be about 2 nm to about 10 nm in length. The nanoparticle core may be about 3 nm to about 30 nm in length. The nanoparticle core may be about 3 nm to about 25 nm in length. The nanoparticle core may be about 3 nm to about 20 nm in length. The nanoparticle core may be about 3 nm to about 15 nm in length. The nanoparticle core may be about 3 nm to about 10 nm in length. The nanoparticle core may be about 3 nm to about 8 nm in length. The nanoparticle core may be about 3 nm to about 6 nm in length.

The nanoparticle core may be about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 nm in length. The nanoparticle core may be about 2, 3, 4, 5, 6, 7, 8, 9, 10, 11, 12, 13, 14, 15, 16, 17, 18, 19, 20, 21, 22, 23, 24, or nm in length. The nanoparticle core may be about 2, 3, 4, 5, 6, 7, 8, 9, 10, 11, 12, 13, 14, 15, 16, 17, 18, 19 or 20 nm in length. The nanoparticle core may be about 2, 3, 4, 5, 6, 7, 8, 9, 10, 11, 12, 13, 14 or 15 nm in length. The nanoparticle core may be about 2, 3, 4, 5, 6, 7, 8, 9 or 10 nm in length. The nanoparticle core may be about 2, 3, 4, 5, 6, 7 or 8 nm in length. The nanoparticle core may be about 3, 4, 5, 6, 7, 8, 9, 10, 11, 12, 13, 14, 15, 16, 17, 18, 19 or 20 nm in length. The nanoparticle core may be about 3, 4, 5, 6, 7, 8, 9, 10, 11, 12, 13, 14 or 15 nm in length. The nanoparticle core may be about 3, 4, 5, 6, 7, 8, 9 or 10 nm in length. The nanoparticle core may be about 3, 4, 5, 6, 7 or 8 nm in length. The nanoparticle core may be about 3, 4, 5, 6, 7, 8, 9 or 10 nm in length. The nanoparticle core may be about 3, 4, 5, 6, 7 or 8 nm in length. The nanoparticle core may be about 3, 4, 5 or 6 nm in length. The nanoparticle core may be about 2 nm in length. The nanoparticle core may be about 3 nm in length. The nanoparticle core may be about 4 nm in length. The nanoparticle core may be about 5 nm in length. The nanoparticle core may be about 6 nm in length. The nanoparticle core may be about 7 nm in length. The nanoparticle core may be about 8 nm in length. The nanoparticle core may be about 9 nm in length. The nanoparticle core may be about 10 nm in length.

The nanoparticle core is typically composed of non-toxic material. The nanoparticle core may be an inorganic nanoparticle core. The inorganic nanoparticle core may be a metal nanoparticle core. When the nanoparticle core is a metal, the metal may be titanium, zirconium, gold, silver, platinum, cerium, arsenic, iron, aluminum or silicon. The metal nanoparticle core may be titanium, zirconium, gold, silver, or platinum and appropriate metal oxides thereof. In embodiments, the nanoparticle core is titanium oxide, zirconium oxide, cerium oxide, arsenic oxide, iron oxide, aluminum oxide, or silicon oxide. The metal oxide nanoparticle core may be titanium oxide or zirconium oxide. The nanoparticle may be titanium. The nanoparticle may be gold. Alternatively, the nanoparticle core may be a polymeric core. Such polymeric cores may include titanium, zirconium, gold, silver, platinum, cerium, arsenic, iron, aluminum or silicon. The polymeric core may include an outer shell layer and an inner shell layer. The outer shell layer may be chemically distinct from the inner shell layer. In embodiments, the outer shell layer and inner shell layer may be a metal such as titanium, gold or silver coated by another metal. Exemplary non-limiting outer and inner shell layer combinations include titanium/gold, titanium/silver, titanium/silicon, gold/titanium, gold/silver, gold/silicon, silicon/titanium, silicon/gold, or silicon/silver. In embodiments, the nanoparticle core includes a combination of metals coated by titanium, zirconium, gold, silver platinum or silicon. The nanoparticle core may be a quantum dot.

In certain embodiments the hydrophilic moiety has the formula:

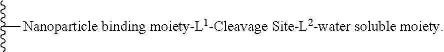

The nanoparticle binding moiety is a chemical linker that connects the nanoparticle core to the cleavage site (e.g. through moiety L¹). In embodiments, the nanoparticle binding moiety is a divalent linker formed by reacting a functional (reactive) group attached to the cleavage site with the nanoparticle core (e.g. a functional (reactive) group on the nanoparticle core). The nanoparticle moiety may be a bond, —S—, —OSi(OR¹)(OR²)(O)—, or —OP(O)(OR³)(O)—. R¹, R², and R³ are independently hydrogen, substituted or unsubstituted alkyl (e.g. C₁-C₅), substituted or unsubstituted heteroalkyl (2 to 5 membered heteroalkyl), substituted or unsubstituted cycloalkyl (3 to 6 membered cycloalkyl), substituted or unsubstituted heterocycloalkyl (2 to 6 membered heterocycloalkyl), substituted or unsubstituted aryl (2 to 6 membered aryl), or substituted or unsubstituted heteroaryl (2 to 6 membered heteroaryl). R¹, R², and R³ may independently be hydrogen, substituted or unsubstituted alkyl (e.g. C₁-C₅). R¹, R², and R³ are independently hydrogen or unsubstituted alkyl (e.g. C₁-C₅).

L¹ and L² are independently a bond, —C(O)—, —O—, —S—, —NH—, —NR⁴—, —C(O)NR⁵—, —S(O)ₙ—, —S(O)NR⁶—, —OP(O)(OR⁷)O—, substituted or unsubstituted alkylene (e.g. C₁-C₁₀), substituted or unsubstituted heteroalkylene (2 to 10 heteroalkylene), substituted or unsubstituted cycloalkylene (3 to 8 membered cycloalkylene), substituted or unsubstituted heterocycloalkylene (3 to 8 membered heterocycloalkylene), substituted or unsubstituted arylene (3 to 8 membered arylene), or substituted or unsubstituted heteroarylene (3 to 8 membered heteroarylene). R⁴, R⁵, R⁶, and R⁷ are independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol n is independently 1 or 2.

L¹ and L² may independently be C₁-C₅ substituted or unsubstituted alkylene (e.g. unsubstituted). L¹ and L² may independently be 3 to 7 membered substituted or unsubstituted heteroalkylene (e.g. unsubstituted). L¹ and L² may independently be 5 to 6 membered substituted or unsubstituted cycloalkylene (e.g. unsubstituted). L¹ and L² may independently be 5 to 6 membered substituted or unsubstituted heterocycloalkylene (e.g. unsubstituted). L¹ and L² may independently be 5 to 6 membered substituted or unsubstituted heterocycloalkylene (e.g. unsubstituted). L¹ and L² may independently be 5 to 6 membered substituted or unsubstituted arylene (e.g. unsubstituted). L¹ and L² may independently be 5 to 6 membered substituted or unsubstituted heteroarylene (e.g. unsubstituted).

The cleavage site of the nanoparticle coating includes a enzymatic cleavage site (e.g. a site cleaved via an enzyme-catalyzed reaction), a metal cleavage site (e.g. a site cleaved via a metal-catalyzed reaction), an acid cleave site (e.g. a site cleaved via an acid-catalyzed reaction), a base cleave site (e.g. a site cleaved via a base-catalyzed reaction), a redox cleavage site (e.g. a site cleaved via a redox-catalyzed reaction), a photo-cleavage site (e.g. a site cleaved via photo-catalyzed reaction), or an electrically cleavage site (e.g. a site cleaved via an electrically-catalyzed reaction). (Wagner, 2012, Chen 2012). In certain embodiments, the nanoparticle coating may include an enzymatic cleavage site, a metal cleavage site, an acid cleavage site, a base cleavage site, a redox cleavage site, or a photo cleavage site. When the cleavage site is an enzymatic cleavage site, the cleavage site may be a peptidase (e.g. a site cleaved by a peptidase) or protease (e.g. a site cleaved by a protease) cleavage site. (Cantley, 2001). In certain embodiments, the cleavage site is a peptidase cleavage site. In certain embodiments the cleavage site recognized by matrix metalloproteinase (MMP). The MMP may be MMP-2 or MMP-9.

In certain embodiments, the cleavage site is covalently linked at the terminal end of the nanoparticle binding moiety and may be cleaved at its contact point with the nanoparticle binding moiety. Alternatively, the cleavage site may be cleaved at its contact point with the water soluble moiety. The cleavage site may be cleaved at some point between its contact points with the nanoparticle binding moiety and the water soluble moiety.

In certain embodiments, the cleavage site is bonded to the nanoparticle core and may be cleaved at its contact point with the nanoparticle core. Alternatively, the cleavage site may be cleaved at its contact point with the water soluble moiety. In certain embodiments, the cleavage site may be cleaved at some point between its contact points with the nanoparticle core and the water soluble moiety.

The water soluble moiety may be a polymer. The water soluble moiety may be a biopolymer moiety (e.g. a polymer of polyscchamides, amino acids, peptides, proteins, nucleic acids, or polynucleotides), alkylpolyaminemoiety (e.g. alkyl having at least two amine (—NH—) moieties, alkylpolyamidemoiety (e.g. alkyl having at least two amide (—NH—C(O)—) moieties, alkylpolyether moiety (e.g. alkyl having at least two ether (—COC—) moieties, alkylpolysulfonatesmoiety (e.g. alkyl having at least two sulfonate (—SO₃) moieties), polyacrylamide moiety (e.g. polymer of —CH₂CHCONH₂— units), carbohydrate moiety, alkylpolyalcohol moiety (e.g. alkyl having at least two alcohol (—OH) moieties. The water soluble moiety may be alkylpolyether, alkylpolyamide, or a biopolymer moiety. The alkylpolyether may be PEG. The water soluble moiety may be poly(vinyl alcohol), poly(lactic-co-glycolic acid), or albumin.

The functionalized nanoparticle may include one or more delivery agents bonded to the nanoparticle core. The delivery agent may be bonded to the nanoparticle core through a cleavable linker. The cleavable linker may be an enzymatic cleavable linker (e.g. a linker cleaved via an enzyme-catalyzed reaction), a metal cleavable linker (e.g. a linker cleaved via a metal-catalyzed reaction), an acid cleavable linker (e.g. a linker cleaved via an acid-catalyzed reaction), a base cleavable linker (e.g. a linker cleaved via a base-catalyzed reaction), a redox cleavable linker (e.g. a linker cleaved via a redox-catalyzed reaction), a photo-cleavable linker (e.g. a linker cleaved via photo-catalyzed reaction), or an electrically cleavable linker (e.g. a linker cleaved via an electrically-catalyzed reaction). When the cleavable linker is an enzymatic cleavable linker, the cleavable linker may be a peptidase cleavable linker (e.g. a linker cleaved by a peptidase) or protease (e.g. a linker cleaved by a protease) cleavable linker. In certain embodiments, the cleavable linker is a peptidase cleavage linker. The linker may be cleaved by matrix metalloproteinase (MMP). The MMP may be MMP-2 or MMP-9. Alternatively, the delivery agent may be encapsulated within the nanoparticle core.

The cleavable linker and the cleavage site may be orthogonal (i.e. one site may be cleaved independently of the other thereby leaving the uncleaved site intact). The orthogonal cleavable linker and cleavage site may be cleaved through the same type of catalysis (e.g. cleaved through enzyme-catalyzed reactions) using different sites or catalysts. Alternatively, the orthogonal cleavable may be cleaved through different types of catalysis (e.g. a cleavable linker cleaved through enzyme-catalyzed reaction and a cleavage site cleaved through metal-catalyzed reaction).

The delivery agent may be a therapeutic agent. Functionalized nanoparticles having therapeutic agents can be used for treatment of a condition responsive to the therapeutic agent. The therapeutic agent may be used for treatment of cancer. The delivery agent may be a labeling agent. Functionalized nanoparticles having labeling agents can be used for detection and/or imaging. For example, the labeling agent may be used to detect or image a tumor site. Alternatively, the labeling agent may be used to trace the passage of the functionalized nanoparticle or a delivery agent attached thereto through the body.

In another aspect, a plurality of functionalized nanoparticles within a vessel is provided. Each functionalized nanoparticle includes a nanoparticle core and a nanoparticle coating. The plurality of nanoparticle cores has an average particle of size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

The nanoparticles, nanoparticle core, and nanoparticle coating are as described above, including all embodiments thereof.

The plurality of functionalized nanoparticles may be supplied as an aqueous suspension or as a powder within the vessel. The vessel may be a storage device, such as an intravenous bag or other readily usable container capable of storing and protecting the functionalized nanoparticles contained within. The vessel may be an administration device, such as a syringe, pen, jet injector, or inhalation device. When the vessel is a syringe, the functionalized nanoparticles may be supplied in a concentration or dose ready for use.

The functionalized nanoparticle may include a therapeutic agent or a labeling agent as provided herein. The therapeutic agent may be bonded to the nanoparticle core through a cleavable linker as provided herein. Likewise, the labeling agent may be bonded to the nanoparticle core through a cleavable linker as provided herein. The cleavable linker and the cleavage site may be orthogonal as provided herein.

In another aspect, a pharmaceutical composition of a plurality of functional nanoparticles is provided. Each functionalized nanoparticle includes a nanoparticle core, a therapeutic agent bonded to the nanoparticle core and a nanoparticle coating. The plurality of nanoparticle cores has an average particle of size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

The nanoparticles, nanoparticle core, and nanoparticle coating are as described above, including all embodiments thereof.

The pharmaceutical composition of the plurality of functionalized nanoparticles may be supplied in an administration device, such as a syringe, pen, jet injector, or inhalation device. The functionalized nanoparticle may include a therapeutic agent or a labeling agent as provided herein. The therapeutic agent may be bonded to the nanoparticle core through a cleavable linker as provided herein. Likewise, the labeling agent may be bonded to the nanoparticle core through a cleavable linker as provided herein. The cleavable linker and the cleavage site may be orthogonal as provided herein.

1. Formulation

The pharmaceutical composition may be prepared and administered in a wide variety of dosage formulations and may be administered orally, rectally, or by injection (e.g. intravenously, intramuscularly, intracutaneously, subcutaneously, intraduodenally, or intraperitoneally). Preparations of pharmaceutical compositions of functionalized nanoparticlesmay include pharmaceutical acceptable carriers that can be either solid or liquid.

Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the functionalize nanoparticles having a therapeutic agent may be mixed with the carrier having the necessary binding properties in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the functionalize nanoparticles having a therapeutic agent with encapsulating material as a carrier providing a capsule in which the functionalize nanoparticles having a therapeutic agent with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the functionalize nanoparticles having a therapeutic agent is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the functionalized nanoparticles provided herein, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the functionalize nanoparticles having a therapeutic agent. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the therapeutic agent used. The composition can, if desired, also contain other compatible therapeutic agents.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

2. Effective Dosages

The pharmaceutical composition may include compositions wherein the therapeutic agent is contained in a therapeutically effective amount, i.e., in an amount effective to achieve its intended purpose. The actual amount effective for a particular application will depend, inter alia, on the condition being treated. For example, when administered in methods to treat cancer, such compositions will contain amounts of therapeutic agent effective to achieve the desired result.

The dosage and frequency (single or multiple doses) of the pharmaceutical composition administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of therapeutic agent by considering factors such as potency, bioavailability, patient body weight, presence and severity of adverse side effects, preferred mode of administration, and the toxicity profile of the selected agent.

3. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Therapeutic agents that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the therapeutic agent is used.

When parenteral application is needed or desired, particularly suitable admixtures for the functionalize nanoparticles having a therapeutic agent included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

Certain embodiments of the functionalized nanoparticles provided herein may be supplied as kits. The kits may further include instruction for use. The kit may supply the nanoparticles provided herein as separate components that may be assembled into functionalized nanoparticles using included instructions.

### III. Methods

In another aspect, a method of treating cancer is provided. The method includes administering a plurality of functionalized nanoparticles within a vessel to a subject in need thereof. Each functionalized nanoparticle includes a nanoparticle core, an anti-cancer agent bonded to the nanoparticle core and a nanoparticle coating. The plurality of nanoparticle cores has an average particle size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site.

The method may include allowing a portion of the functionalized nanoparticles to localize to a cancer cell and allowing a cleaving agent to cleave the cleavage site (e.g. one or more cleavage sites) of at least one of the functionalized nanoparticles within the portion. The cleaving thereby removes the nanoparticle coating and forms a denuded anti-cancer nanoparticle.

The plurality of functionalized nanoparticles may be administered in a wide variety of dosage formulations and may be administered orally, rectally, or by injection (e.g. intravenously, intramuscularly, intracutaneously, subcutaneously, intraduodenally, or intraperitoneally) as provided herein. In certain embodiments, the plurality of functionalized nanoparticles is administered by injection using a vessel such as a syringe, pen, or jet injector.

The nanoparticles, nanoparticle core, and nanoparticle coating are as described above, including all embodiments thereof.

The anti-cancer agent may be bonded to the nanoparticle core through a cleavable linker. The cleavable linker may be an enzymatic cleavable linker (e.g. cleaved through enzyme-catalyzed reaction), a metal cleavable linker (e.g. cleave through a metal-catalyzed reaction), an acid cleavable linker (e.g. cleaved through an acid-catalyzed reaction), a base cleavable linker (e.g. cleaved through a base-catalyzed reaction), a redox cleavable linker (e.g. cleaved through a redox-catalyzed reaction), a photo-cleavable linker (e.g. cleaved through photo-catalyzed reaction), or an electrically cleavable linker (e.g. cleavage through an electrically-catalyzed reaction). When the cleavable linker is an enzymatic cleavable linker, the cleavable linker may be a peptidase (e.g. cleaved by a peptidase) or protease (e.g. cleaved by a protease) cleavage site. In certain embodiments, the cleavage site is a peptidase cleavage site. In certain embodiments, the cleavage site is cleaved by matrix metalloproteinase (MMP). The MMP may be MMP-2 or MMP-9. Alternatively, the anti-cancer agent may be encapsulated within the nanoparticle core.

In certain embodiments, the cancer cell forms part of a tumor, wherein the cleaving agent (e.g. a protease) may be located proximal to the cancer cell. The functionalized nanoparticles may be cleaved at the cleavage site of the plurality of hydrophilic moieties, thereby removing the nanoparticle coating and forming a denuded anti-cancer nanoparticle. In certain embodiments the cleavable linker of the anti-cancer agent may be cleaved by a cleaving agent thereby releasing the anti-cancer agent. The cleaving agent may be a peptidase or a protease. The cleaving agent may be matrix metalloproteinase (MMP). The MMP may be MMP-2 or MMP-9. Alternatively, the denuded anti-cancer nanoparticle may act as the therapeutic agent to treat the cancer.

In another aspect, a method of detecting a cancer cell within a subject is provided. The method includes administering a plurality of functionalized nanoparticles within a vessel to a subject in need thereof. Each functionalized nanoparticle includes a nanoparticle core and a nanoparticle coating. The plurality of nanoparticle cores has an average particle of size of about 2 to about 35 nm. Each of the nanoparticle coatings includes a plurality of hydrophilic moieties bonded to the nanoparticle core. Each of the hydrophilic moieties includes a nanoparticle binding moiety bonded to the nanoparticle core, a cleavage site covalently linked to the nanoparticle binding moiety, and a water soluble moiety covalently linked to the cleavage site. The method includes allowing a portion of the functionalized nanoparticles to localize to the cancer cell and allowing a cleaving agent to cleave the cleavage site of at least one of the portion of the functionalized nanoparticles thereby removing the nanoparticle coating and forming a denuded detectable nanoparticle. The method further includes detecting the denuded detectable nanoparticle thereby detecting the cancer cell.

The plurality of functionalized nanoparticles may be administered in a wide variety of dosage formulations and may be administered orally, rectally, or by injection (e.g. intravenously, intramuscularly, intracutaneously, subcutaneously, intraduodenally, or intraperitoneally) as provided herein. In certain embodiments, the plurality of functionalized nanoparticles is administered by injection using a vessel such as a syringe, pen, or jet injector.

The nanoparticles, nanoparticle core, and nanoparticle coating are as described above, including all embodiments thereof

The functionalized nanoparticles may further include a cancer cell binding agent bonded to the nanoparticle core. The cancer cell binding agent may localized the functionalized nanoparticles to particular cancer cells. In certain embodiments, the cancer cell binding agent recognizes or is recognized by a recognition element. The recognition element may be a peptide sequence, a nucleic acid sequences, or a small molecule. The recognition element may for example be specific for an enzyme, an antibody, a substrate, a particular cell type or a particular condition unique to a cellular microenvironment such as a change in temperature. The recognition element may localize the functionalized nanoparticles to the cancer cell via affinity binding between the recognition element and the specific enzyme, antibody, substrate, or particular cell type.

In certain embodiments, the cancer cell forms part of a tumor, wherein the cleaving agent (e.g. a peptidase or a protease) may be located proximal to the cancer cell. The functionalized nanoparticles may be cleaved at the cleavage site, thereby removing the nanoparticle coating and forming a denuded anti-cancer nanoparticle. In certain embodiments the cleavable linker of the anti-cancer agent may cleaved by a cleaving agent thereby releasing the anti-cancer agent. Alternatively, the denuded anti-cancer nanoparticle may act as the therapeutic agent to treat the cancer.

The nanoparticle may further include a labeling agent bonded to the nanoparticle core. The detection of the denuded nanoparticle may be performed by detecting the labeling agent. The labeling agent may be a fluorophore moiety, a radio-labeled moiety, or a magnetic contrast agent. The labeling agent may be for example, a fluorescent protein such as GFP or fluorescein. Alternatively, the labeling agent may be for example Fluorine-18, Gallium-68, or Copper-64. When the labeling agent is a magnetic contrast agent, the labeling agent may be for example gadolinium, iron oxide, iron platinum, manganese, or a paramagnetic species.

### IV. Examples

### Example 1

### Functionalization of Gold Nanoparticles

Well characterized 5 nm citrate stabilized AuNPs (TedPella, Redding, Calif.) were functionalized using various recognition element/blocker element moieties by simple ligand exchange. Recognition element/blocker element moieties were PEG-PVGLIGC (PEG-SEQ ID NO:1), PEG-GIVGPLC (PEG-SEQ ID NO:2), LIGC (SEQ ID NO:3), and PEG-SH (all peptides from CS Bio, Menlo Park, Calif.). SEQ ID NO:1 is a peptide sequence cleaved by both MMP-2 and MMP-9, while SEQ ID NO:2 is a scrambled sequence that is not recognized by either protease (Turk 2001; Chau 2004). SEQ ID NO:3 represents the MMP-2/MMP-9 cleavage product of SEQ ID NO:1. The resultant AuNP conjugates were AuNP-PVGLIGC-PEG (cleavable), AuNP-GIVGPLC-PEG (noncleavable negative control), AuNP-LIGC (denuded positive control), and PEG-AuNP (PEG blocker control). For functionalization, 500 μg of the dry ligand moiety was added into 200 μL of the filtered gold nanoparticles (632 μg/mL) in suspension and mixed well overnight at room temperature on a laboratory platform rocker (Barnstead Int., Dubuque, 1A) at a speed of 4 cycles/minute.

### Example 2

### Purification of Functionalized Gold Nanoparticles

After overnight incubation on the rocker, conjugates were spun down by table top centrifugation (15,000 rpm, 1 hour). The solution was removed and Milli-Q water was added, and the solution was vigorously shaken by hand and then centrifuged. These steps were repeated three times. After the third wash, the particles were suspended in Milli-Q water and dialyzed against 100 mL of Milli-Q water for 4 hours (the water was replaced every 30 minutes) using 1000 Da cut off membrane dialysis tubing.

### Example 3

### Cleavage of Functionalized Gold Nanoparticles

All four purified functionalized conjugates from Example 2 were subjected to cleavage by human matrix metalloproteinase (MMP-2) (Abeam, Cambridge, Mass.) as instructed by the manufacturers. Briefly, the conjugates were subjected to centrifugation (15,000 rpm, 1 hour), and the resultant AuNP pellets were suspended in 200 μL of 100 mM Tris buffer pH 7.2 containing 0.15 M NaCl and 5 mM CaCh. Active MMP-2 (0.5 μL, 0.25 μg/μL) was added to this suspension to achieve a final concentration of 0.625 μg/mL to initiate the cleavage reaction, followed by incubation at 37° C. for 16 hours. Successful cleavage was monitored by visual inspection, with cleavage resulting in the change of suspension from red to purple. The conjugate PEG-PVGLIGC-AuNP was also cleaved using trypsin with the same protocol. After 16 hours, the resulting particles were purified as described above.

### Example 4

### Physical Characterization of Functionalized Gold Nanoparticles

Dynamic light scattering (DLS) and zeta potential measurements were performed on a Brookhaven 90 Plus/BI-MAS Instrument (Brookhaven Instruments, New York). DLS measurements indicated that the blocker control, intact cleavable (PEG-PVGLIGC-AuNP), and non-cleavable (PEGGIVGPLC-AuNP) AuNPs were of similar size (22±1 nm, 26±4 nm, and 19±2 nm, respectively), while the denuded control AuNPs (LIGC-AuNP) aggregated to give an apparent hydrodynamic radius of 258±8 nm. Following exposure to MMP-2, the apparent hydrodynamic radius of the cleavable AuNP rapidly shifted to 311±4 nm, suggested that the PEG blocking element had been removed and that the particles had aggregated. The non-cleavable AuNP, on the other hand, did not exhibit a significant change following MMP-2 exposure. Zeta potential measurements likewise indicated that the cleavable AuNPs were similar to the non-cleavable control AuNPs prior to MMP-2 treatment and similar to the denuded control AuNPs after MMP-2 treatment.

UV-vis and fluorescence spectroscopy measurements were recorded on a BioTek Synergy 2 micro plate reader (BioTek® Instruments Inc, Vermont, PN) operated at a resolution of 1 nm using 96-well polystyrene transparent plates (Becton Dickinson Labware, Franklin Lakes, N.J.). Prior to MMP-2 exposure, the cleavable AuNPs had a λₘₐₓ very similar to that of AuNPs alone. After exposure to MMP-2, the λₘₐₓ was red shifted and was very similar to that of the denuded control AuNPs. This phenomenon was visible to the naked eye, and occurred very rapidly. The red shift was indicative of particle aggregation.

Confocal transmission electron microscopy (TEM) images were obtained using a Zeiss LSM 710 confocal laser-scanning microscope with a Plan-Apochromat 20×/0.8 objective (Carl Zeiss Microimaging, Thornwood, N.Y.). Optical sections were collected at 1 μm spacing and shown as a maximum intensity projection using Zen 2009 software (Carl Zeiss). TEM images of the cleavable AuNPs prior to MMP-2 treatment showed well dispersed particles, while images after MMP-2 treatment showed large aggregates similar to those seen with denuded control.

Field flow fractionation (FFF) was performed using an Eclipse 3 (Wyatt Technology Corp., Santa Barbara, Calif.) with an Agilent 1260 HPLC system. The channel was equipped with a 5 kDa molecular weight cutoff polyethersulfone membrane. A 350 μm thick spacer was used that was 240 mm from injection site to outlet, 21.5 mm wide at the injection site and 6 mm wide at the outlet site. The mobile phase consisted of sterile saline and the elution was monitored at 530 nm by Agilent UVdiode array detector. The detailed control of solvent flow is shown below:

| Start | End | Cross Flow at | Cross Flow | | Detector | |
|---|---|---|---|---|---|---|
| Time | Time | Start Time | at End Time | Focus Flow | Flow | Inject Flow |
| [min] | [min] | [mL/min] | [mL/min] | [mL/min] | [mL/min] | [mL/min] |
| 0 | 1 | 0 | 0 | 0 | 1 | — |
| 1 | 2 | — | — | 1.5 | 1 | — |
| 2 | 4 | — | — | 1.5 | 1 | 0.2 |
| 4 | 5 | — | — | 1.5 | 1 | — |
| 5 | 20 | 3 | 0 | — | 1 | — |
| 20 | 25 | 0 | 0 | — | 1 | — |
| 25 | 30 | 0 | 0 | — | 1 | 0.2 |

FFF confirmed that cleavable AuNPs behaved similarly in solution to blocker control AuNPs prior to MMP-2 treatment, but behaved more like denuded control AuNPs after MMP-2 treatment.

All of these results suggest that cleavable AuNPs were converted to denuded AuNPs following exposure to MMP-2. Non-cleavable control and blocker control AuNPs exhibited no cleavage. Cleavable AuNPs treated with trypsin did not exhibit cleavage, confirming the specificity of the cleavage reaction.

| | Particle core | Hydrodynamic | Zeta | |
|---|---|---|---|---|
| Nanoparticle | mean diameter | diameter by DLS | Potential | λₘₐₓ |
| sample | by TEM (nm) | (nm) | (mV) | (nm) |
| AuNP | 4 ± 1 | 10 ± 4 | −14 ± 2 | 519 |
| PEG-AuNPs | 4 ± 1 | 22 ± 1 | −17 ± 1 | 522 |
| Cleavable | 4 ± 1 | 26 ± 4 | −9 ± 3 | 525 |
| AuNPs, before | | | | |
| MMP-2 | | | | |
| Cleavable | 4 ± 1 | 311 ± 4 | −45 ± 4 | 553 |
| AuNPs, after | | | | |
| MMP-2 | | | | |
| Non-Cleavable | 4 ± 1 | 19 ± 2 | −5 ± 2 | 525 |
| AuNPs, before | | | | |
| MMP-2 | | | | |
| Non-Cleavable | 4 ± 1 | 23 ± 4 | −6 ± 2 | 526 |
| AuNPs, after | | | | |
| MMP-2 | | | | |
| Denuded AuNPs | 4 ± 1 | 258 ± 8 | −39 ± 2 | 550 |

### Example 5

### Cytotoxicity of Functionalized Gold Nanoparticles

PEGylated AuNPs have generally been observed to be non-toxic (Khan 2007; Patra 2007). Cytotoxicity assessment in human breast adenocarcinoma (MDA-MB-231, ATCC) and normal fibroblast (GM 00673J, Coriell Institute for Medical Research, Camden, N.J.) cell lineswas performed using the MTS assay CellTiter 96® Aqueous cell proliferation assay (Promega, Madison Wis.) and LIVE/DEAD staining MTS (3-(4,5-dimethylthiazol-2-yl)-5-(3-carboxymethoxyphenyl)-2-(4-sulfophenyl)-2H-tetrazolium) is a tetrazolium compound that gets converted into a colored formazan by the living cells.

For the MTS assay, cells seeded at 3×10⁴ per well in a 96-well tissue culture plate were grown to 80% confluence in 200 μL of Roswell Park Memorial Institute medium (RPMI) supplemented with 0.2 mM L-glutamine, 100 U ml⁻¹ penicillin, and 10% FBS for breast adenocarcinoma cells and Eagles minimum essential medium (EMEM) supplemented with 100 U ml⁻¹ penicillin and 10% FBS for fibroblast cells under a 5% CO2 humidified atmosphere at 37° C. For each treatment, the cells were washed twice with PBS and then incubated with various conjugates for 12 hours. Untreated cells and cells treated with AuNPs separately served as controls. For statistical data analysis and to ensure reproducibility, each treatment was performed in octuplet, and each assay was repeated at least three times. After exposure to the various AuNPs, the cells were rinsed once with PBS. 200 μL of supplemented RPMI was added, and the plate was incubated at 37° C. for 18-24 hours. The MTS reagent (20 μL) was then added to each well and the cells were re-incubated at 37° C. for an additional 3 hours, followed by absorbance measurements at 490 nm using a spectrophotometer. The quantity of formazan produced as determined by the absorbance is directly proportional to the number of live cells. Both cleavable and denuded control AuNPs caused slightly reduced cell viability as measured by MTS.

For LIVE/DEAD staining, cells were cultured and treated with various AuNPs as described above for the MTS assay. Following nanoparticle exposure, the cells were stained with calcein and ethidium homodimer (EthD-1) (Invitrogen). The dyes were mixed together and appropriately diluted so that the effective working solution contains 2 μM of calcein and 4 μM of EthD-1 which is then directly added to cells following manufacturer's protocol. After one hour of incubation with the dye, the cells were washed two times with sterile PBS, treated with fixative (4% paraformaldehyde in PBS) for 30 min followed by a couple of washes with PBS prior to assessing the membrane integrity using confocal microscopy. The excitation was at 485 nm and the emission at 515 and 635 nm.

### Example 6

### Cell Uptake of Functionalized Gold Nanoparticles

Dark-field microscopy was performed to image AuNP uptake over a relatively large area. Cells were grown to 80% confluence on a glass cover slip placed inside the regular cell culture dishes under physiological conditions for 24-48 hours. Cells were treated with various conjugates as described above in Example 5, except regular growth medium was replaced with low nutrient minimal medium (OPTI-MEM) to prevent enriched medium mediated aggregation of particles (Suresh 2012). After 12 hours of exposure, excess medium was removed, and the cells were washed twice with PBS. Cover slips were carefully taken from the dishes using a sharp edge forcep and allowed to dry. A drop of oil was put on the cover slip, which was mounted onto the glass slide and adhered using nail polish to prevent drying. Slides and cover slips were of premium quality with non-corrosive and non-fogging properties (Fisher Scientific). Hyperspectral imaging was performed using a CytoViva dark field microscope system equipped with CytoViva Hyperspectral Imaging System 1.2. Spectral analyses were performed using customized ENVI hyperspectral analysis software provided by the manufacturers.

To establish what hyperspectral signal to map in the experimental samples, AuNPs were imaged and a hyperspectral spectra was recorded for each pixel in the image. A collection of spectra was then obtained by selecting pixels corresponding to the AuNPs. To assess AuNP uptake and distribution, dark-field images were obtained of MDA-MB-231 adenocarcinoma cells exposed to cleavable AuNPs before and after MMP-2 exposure and denuded control AuNPs. Mapping was performed on these images to false-color each pixel red that matched one of the hyperspectral spectra from the AuNP sample. Prior to MMP-2 exposure, the cleavable AuNPs exhibited no uptake. After MMP-2 exposure, the cleavable AuNPs exhibited robust uptake similar to denuded control AuNPs. Non-cleavable and blocker control AuNPs exhibited no uptake under any conditions. The stark difference in uptake for the cleavable AuNPs before and after MMP-2 exposure demonstrates the proof-of-principle that denuding of NPs can be used to target their uptake.

For TEM experiments, cells treated with various conjugates as described above were collected by treating with 0.2% trypsin-EDTA, followed by washing with PBS and table top centrifugation (1000 rpm, 5 min), fixed with 2% glutaraldehyde in 0.1 M Cacodylate buffer (Na(CH₃)₂AsO₂.3H₂O), pH 7.2, at 4° C. overnight. The following day the cells were washed three times with 0.1 M Cacodylate buffer, post-fixed with 1% OsO₄ in 0.1 M Cacodylate buffer for 30 min and washed three times with 0.1 M Cacodylate buffer. Samples were then dehydrated using 60%, 70%, 80%, 95% ethanol and 100% absolute ethanol (twice), propylene oxide (twice), and were left in propylene oxide/Eponate (1:1) overnight at room temperature under sealed environment. The following day the vials were left open until the propylene oxide evaporated (˜2-3 hours). Samples were infiltrated with 100% Eponate and polymerized at ˜64° C. for 48 hours. Ultra-thin sections (˜70 nm thick) were cut using a Leica Ultra cut UCT ultra-microtome equipped with a diamond knife, and the sections were picked up on 200 mesh copper EM grids. The grids were stained with 2% uranyl acetate for 10 minutes, followed by Reynold's lead citrate staining for a minute and were imaged using a FEI Tecnai 12 TEM equipped with a Gatan Ultrascan 2K CCD camera at an accelerating voltage of 1200 kV.

In agreement with the dark-field imaging and hyperspectral mapping, no uptake was observed for cleavable AuNPs prior to MMP-2 treatment, blocker control AuNPs, or non-cleavable AuNPs. Cleavable AuNPs exposed to MMP-2, on the other hand, exhibited dramatic uptake was seen for cleavable AuNPs exposed to MMP-2 and for the denuded positive control. In both cases, aggregates of AuNPs were observed in various vesicular bodies throughout the cell, with no evidence of uptake by the nucleus. Occasionally, a few scattered nanoparticle aggregates were also observed in the cytoplasm. The TEM data strongly suggests that after removal of the PEG blocking element, the resulting denuded AuNPs are taken up by endocytosis.

### V. References

Arap Science 279:377 (1998)
Azzam J Natl Cancer Inst 85:1758 (1993)
Berlin ACS Nano 4:4621 (2010)
Bhirde ACS Nano 3:307 (2009)
Chau Bioconjug Chern 15:931 (2004)
Choi Nat Biotechnol 25:1165 (2007)
Davies Cancer Res 53:5365 (1993)
Donaldson Cancer Biol Ther 8:2147 (2009)
El-Sayed Cancer Lett 239:129 (2006)
Fay Immunotherapy 3:381 (2011)
Ferrar Nat Rev Cancer 5: 161 (2005)
Giambemardi Matrix Biol 16:483 (1998)
Harrington Br J Cancer 83:232 (2000)
Harris Angew Chem Int Ed Engl 45:3161 (2006)
Hutter ACS Nano 4:2595 (2010)
Jain Nano Today 2:18 (2007)
Khan Chembiochem 8:1237 (2007)
Mu ACS Nano 4:15ll (2010)
Olson Proc Natl Acad Sci USA 107:43ll (2010)
Paciotti Clin Cancer Res 7:3673 S (2001)
Paciotti Drug Deliv ll:169 (2004)
Paciotti Drug Develop Res 67:47 (2006)
Patra N anomedicine 3: 111 (2007)
Riehemann Angew Chem Int Ed Engl 48:872 (2009)
Steams Cancer Res 53:878 (1993)
Suresh Langmuir 28:2727 (2012)
Turk Nat Biotechnol 19:661 (2001)
Vasir Technol Cancer Res Treat 4:363 (2005)
Verma Small 6:12 (2010)
Zhang Nano Lett 6:1988 (2006)
Wagner Bioorg Med Chem 20:571 (2012)
Cantley Nat Biotech 19:662 (2001)
Chen Bioconjugate Chem 23:705 (2012)

1. A functionalized nanoparticle comprising a nanoparticle core and a nanoparticle coating, wherein:
(i) said nanoparticle core is about 2 to about 35 nm in length;
(ii) said nanoparticle coating comprises a plurality of hydrophilic moieties bonded to said nanoparticle core, wherein each of said hydrophilic moieties comprise:
(a) a nanoparticle binding moiety bonded to said nanoparticle core;
(b) a cleavage site covalently linked to said nanoparticle binding moiety; and
(c) a water soluble moiety covalently linked to said cleavage site.

2. The functionalized nanoparticle of claim 1, wherein said water soluble moiety is a water soluble polymer moiety.

3. The functionalized nanoparticle of claim 1, wherein said nanoparticle core is about 3 to about 10 nm in length.

4. The functionalized nanoparticle of claim 1, wherein said nanoparticle core is an inorganic nanoparticle core.

5. The functionalized nanoparticle of claim 1, wherein said nanoparticle core is a metal nanoparticle core.

6. The functionalized nanoparticle of claim 5, wherein said metal nanoparticle core comprises titanium, zirconium, gold, silver, platinum, cerium, arsenic, iron, aluminum or silicon.

7. The functionalized nanoparticle of claim 1, wherein said nanoparticle core is a polymeric core.

8. The functionalized nanoparticle of claim 1, wherein said nanoparticle core comprises an outer shell layer and an inner layer, wherein said outer shell layer is chemically distinct from said inner layer.

9. The functionalized nanoparticle of claim 1, further comprising a delivery agent bonded to said nanoparticle core.

10. The functionalized nanoparticle of claim 1, wherein said delivery agent is bonded to said nanoparticle core through a cleavable linker.

11. The functionalized nanoparticle of claim 10, wherein said cleavable linker is an enzymatic cleavable linker, a metal cleavable linker, an acid cleavable linker, a basic cleavable linker, a redox cleavable linker, a photo cleavable linker, or an electrically cleavable linker.

12. The functionalized nanoparticle of claim 10, wherein said cleavable linker and said cleavage site are orthogonally cleavable elements.

13. The functionalized nanoparticle of claim 9, wherein said delivery agent is a therapeutic agent or a labeling agent.

14. The functionalized nanoparticle of claim 1, wherein said water soluble moiety is a biopolymer moiety, alkylpolyaminemoiety, alkylpolyamidemoiety, alkylpolyether moiety, alkylpolysulfonatesmoiety, polyacrylamide moiety, carbohydrate moiety, alkylpolyalcohol moiety.

15. The functionalized nanoparticle of claim 14, wherein said alkylpolyether moiety is a PEG moiety.

16. The functionalized nanoparticle of claim 1, wherein said cleavage site is an enzymatic cleavage site, a metal cleavage site, an acid cleavage site, a basic cleavage site, a redox cleavage site, a photo cleavage site, or an electrically cleavage site.

17. The functionalized nanoparticle of claim 16, wherein said enzymatic cleavage site is a peptidase cleavage site.

18. A plurality of functionalized nanoparticles within a vessel, wherein each functionalized nanoparticle comprises a nanoparticle core and a nanoparticle coating, wherein:
(i) said plurality of nanoparticle cores have an average particle size of about 2 to about 35 nm;
(ii) each of said nanoparticle coatings comprises a plurality of hydrophilic moieties bonded to said nanoparticle core, wherein each of said hydrophilic moieties comprise:
(a) a nanoparticle binding moiety bonded to said nanoparticle core;
(b) a cleavage site covalently linked to said nanoparticle binding moiety; and
(c) a water soluble moiety covalently linked to said cleavage site.

19. The plurality of functionalized nanoparticles of claim 18, wherein said vessel is an administration device.

20. A pharmaceutical composition comprising plurality of functionalized nanoparticles, wherein each functionalized nanoparticle comprises a nanoparticle core, a therapeutic agent bonded to said nanoparticle core and a nanoparticle coating, wherein:
(i) said plurality of nanoparticle cores have an average particle size of about 2 to about 35 nm;
(ii) each of said nanoparticle coatings comprises a plurality of hydrophilic moieties bonded to said nanoparticle core, wherein each of said hydrophilic moieties comprise:
(a) a nanoparticle binding moiety bonded to said nanoparticle core;
(b) a cleavage site covalently linked to said nanoparticle binding moiety; and
(c) a water soluble moiety covalently linked to said cleavage site.

21. A method of treating cancer, said method comprising administering a plurality of functionalized nanoparticles within a vessel to a subject in need thereof, wherein each functionalized nanoparticle comprises a nanoparticle core, an anti-cancer agent bonded to said nanoparticle core and a nanoparticle coating, wherein:
(i) said plurality of nanoparticle cores have an average particle size of about 2 to about 35 nm;
(ii) each of said nanoparticle coatings comprises a plurality of hydrophilic moieties bonded to said nanoparticle core, wherein each of said hydrophilic moieties comprise:
(a) a nanoparticle binding moiety bonded to said nanoparticle core;
(b) a cleavage site covalently linked to said nanoparticle binding moiety; and
(c) a water soluble moiety covalently linked to said cleavage site.

22. The method of claim 21, further comprising
(1) allowing a portion of said functionalized nanoparticles to localize to a cancer cell; and
(2) allowing a cleaving agent to cleave the cleavage site of at least one of said portion of said functionalized nanoparticles thereby removing the nanoparticle coating and forming a denuded anti-cancer nanoparticle.

23. The method of claim 22, wherein said cleaving agent is a protease.

24. A method of detecting a cancer cell within a subject, said method comprising:
(i) administering a plurality of functionalized nanoparticles within a vessel to a subject in need thereof, wherein each functionalized nanoparticle comprises a nanoparticle core and a nanoparticle coating, wherein:
(a) said plurality of nanoparticle cores have an average particle size of about 2 to about 35 nm;
(b) each of said nanoparticle coatings comprises a plurality of hydrophilic moieties bonded to said nanoparticle core, wherein each of said hydrophilic moieties comprises:
(1) a nanoparticle binding moiety bonded to said nanoparticle core;
(2) a cleavage site covalently linked to said nanoparticle binding moiety; and
(3) a water soluble moiety covalently linked to said cleavage site;
(ii) allowing a portion of said functionalized nanoparticles to localize to said cancer cell;
(iii) allowing a cleaving agent to cleave the cleavage site of at least one of said functionalized nanoparticles of said portion thereby removing the nanoparticle coating and forming a denuded detectable nanoparticle; and
(iv) detecting said denuded detectable nanoparticle thereby detecting said cancer cell.

25. The method of claim 24, wherein said cleaving agent is specifically localized to said cancer cell.

26. The method of claims 24, wherein said nanoparticle further comprises a labeling agent bonded to said nanoparticle core and wherein said detecting comprises detecting said labeling agent.

27. The method of claim 24, wherein said nanoparticle further comprises a cancer cell binding agent bonded to said nanoparticle core.

28. The method of claims 24, wherein said cleaving agent is a protease.
